(19) United States
(12) Patent Application Publication
(10) Pub. No.: US 2026/0256370 A1
(43) Pub. Date: Sep. 3, 2026

(54) METHOD, DEVICE AND SYSTEM FOR VASCULAR BLOOD FLOW MEASUREMENT

(71) Applicant: University of South Alabama, Mobile, AL (US)

(72) Inventors: David Arthur Nelson, Daphne, AL (US); Christopher Michael Francis, Mobile, AL (US); Saeed Iftakhar Latif, Mobile, AL (US)

(21) Appl. No.: 19/544,031

(22) Filed: Feb. 19, 2026

Related U.S. Application Data
(60) Provisional application No. 63/764,627, filed on Feb. 28, 2025.

(51) Int. Cl. A61B 5/026; A61B 5/01

(52) U.S. Cl. CPC A61B 5/026; A61B 5/01; A61B 2562/16

(57) ABSTRACT

A method for determining a mammalian vascular blood flow metric is described. The method of the present invention includes the steps of (a) heating a target skin area by irradiating the target skin area for a heating test period with radio frequency energy, the radio frequency energy including a plurality of radio frequency beams; (b) measuring the temperature of the target skin area periodically during a measurement period to create a temperature data set; (c) determining at least one temperature change time constant based on the temperature data set; and (d) calculating a blood flow metric correlating to the temperature change time constant. A device and system for determining a mammalian vascular blood flow metric is also disclosed. The present invention may be useful in the assessment of human physical health and fitness as well as the diagnoses, monitoring and treatment of disease and therapeutic vascular interventions.

### FIELD OF THE INVENTION

The present invention relates broadly to the field of mammalian (e.g., human and animal) health and more particularly to methods, devices and systems for mammalian health assessment and monitoring as well as disease diagnosis and treatment.

### BACKGROUND OF THE INVENTION

Vascular (describing vessel diameters greater than or equal to 0.1 mm) and microvascular (describing vessel diameters less than 0.1 mm) blood flow is critical as a general matter to overall mammalian, and more particularly human, fitness and health. Accordingly, quantitative measurement of blood flow in the skin, as well as blood flow rate of change, provides medical professionals with an important criterion to assess patients' physical condition including overall circulatory health as well as a useful tool for screening and detection of various mammalian disorders. For example, skin blood flow rate may be useful in the assessment of medical conditions and management of chronic diseases such as continuing assessment of peripheral circulation in diabetic patients, peripheral vascular disease, ovarian hyperstimulation syndrome, Raynaud's disease, and loss of thermoregulatory function associated with aging and spaceflight. The measurement of skin blood flow rate can also be used to assess emergent medical conditions, such as for example circulatory shock and heat stroke. The measurement of skin blood flow rate can also be used to assist medical personnel in pre-surgical determination of amputation levels and post-surgical evaluations of arterial patency of skin flaps or revascularization therapies. For ease and simplicity, the term “vascular” as used herein is intended to describe blood vessel flow generally, expressly including without limitation blood flow in microvascular and vascular categories.

Millimeter Wave (MMW) flow sensing uses a radio frequency (RF) energy source to produce mild heating of human or animal skin, with the resulting skin temperature correlated to blood flow in the underlying microvasculature through application of Fick's Principle.

As described in U.S. Pat. No. 4,228,805, the rate of blood flow within a tissue can be measured by a process of heating a device, such as a probe, which is in contact with the tissue being examined, and then recording the temperature changes by a thermocouple positioned in or near the probe. The recorded temperature changes are representative of the blood flow in the tissue. The heated device and thermocouple effectively act as a flowmeter for determining the blood flow as a function of the rate at which heat is carried away from the tissue.

The art was advanced by the development of a method, system and non-contact, non-invasive device for calculating a blood flow rate in an area as described in U.S. Pat. No. 7,267,651, invented by a co-inventor of the invention of the present application. The patented '651 method includes generating an electrical signal having a power output, irradiating an area with the electrical signal, determining an incident power density of the electrical signal as a function of the power output and dimensions of the area being irradiated, heating the area in response to irradiating the area, sensing the temperature of the area within a time period, calculating the rate of temperature change of the area, and calculating the blood flow rate in the area based on the power density of the electrical signal. The contents and disclosure of U.S. Pat. No. 7,267,651 are expressly incorporated herein by reference.

The art was again advanced by the development of a method of, and an improved apparatus for, skin blood flow measurement as described in U.S. Pat. No. 11,293,793, co-invented by two of the co-inventors of the invention described in the present application. The method includes the steps of: (a) generating an electrical signal having a power output; (b) irradiating an area of skin surface with the electrical signal; (c) determining an incident power density of the electrical signal as a function of the power output and dimensions of the area being irradiated; (d) heating the area in response to irradiating the area; (e) sensing the temperature of the area within a time period; calculating the temperature change of the area; and (f) calculating the blood flow rate in the area based on the power density of the electrical signal and the temperature change and/or the rate of temperature change of the area; wherein the electrical signal includes a frequency in the RF band. The patented '793 apparatus generally includes a chassis; an antenna such as a microstrip or patch antenna connected with the chassis such that the antenna directs radio frequency (RF) energy at an area of a skin surface below the chassis; a temperature sensor also connected with the chassis such that the temperature sensor is held in fixed relation to the antenna and such that the temperature sensor is also directed to the same said area of a skin surface below the chassis for measurement of temperature of the area. The chassis supports the antenna and the temperature sensor in fixed relation to the subject skin surface area. An analysis module can calculate blood flow rate at the target surface based on temperature (or temperature rate of change) and the power density value. The contents and disclosure of U.S. Pat. No. 11,293,793 are expressly incorporated herein by reference.

The inventions described in the '651 and '793 patents have provided the medical field with a robust, reliable and accurate means for determining blood flow rate and rate of change data in support of human health assessment and diagnostic work for many years. Nonetheless, they have drawbacks. For example, they require transmitter power measurement to support power density calculation, which in turn may require difficult and time-consuming device re-calibration on a frequent basis. Further, their function is based primarily on the power absorbed by the skin, which in turn depends on variables which are difficult to determine, such as (i) transmitter output (power, wavelength), antenna pattern and orientation with respect to the target skin area; (ii) skin tissue absorptivity, which depends on frequency, permittivity of the tissue (at the given frequency), tissue hydration, and other biological and physiological variables which may be subject-dependent-all of which are difficult if not impossible to control even if precise calibration could be achieved with every use. Accordingly, a continuing unmet need exists for improved, user-friendly vascular blood flow rate measurement methods, devices and systems with even higher data accuracy and repeatability.

All terms used herein are given their common meaning to those of ordinary skill in the art unless otherwise defined or described herein.

### SUMMARY OF THE INVENTION

In one aspect, the present invention relates to a method for determining a mammalian vascular blood flow metric. The method of the present invention includes the steps of (a) heating a target skin area by irradiating said target skin area for a heating test period with radio frequency energy, said energy comprising a plurality of radio frequency beams; (b) measuring the temperature of said target skin area periodically during a measurement period to create a temperature data set; (c) determining at least one temperature change time constant based on the temperature data set; and (d) calculating a blood flow metric correlating to said temperature change time constant.

In yet another aspect, the present invention relates to a device for determining a mammalian vascular blood flow metric at a target skin area. In this aspect, the device of the present invention includes: a. at least one support; b. an antenna array including a plurality of radio frequency energy beam-emitting antennas mounted on said support and aligned to direct radio frequency energy at and heat at least a portion of said target skin area; and c. a temperature sensor array comprising one or more temperature sensors mounted on said support and aligned to receive electromagnetic emissions from at least a portion of said target skin area; wherein said antenna array and said temperature sensor array are (a) mounted in fixed relation to said target skin area and (b) do not contact said target skin area.

In still another aspect, the present invention is directed to a system for measuring mammalian vascular blood flow rate at a target skin area. In this aspect, the system of the present invention includes (i) a device for determining a mammalian vascular blood flow metric at a target skin area, the device including a. at least one support; b. an antenna array including a plurality of radio frequency beam-emitting antennas mounted on the support and aligned to direct radio frequency energy at and heat at least a portion of said target skin area; c. a temperature sensor array comprising one or more temperature sensors mounted on the support and aligned to receive electromagnetic emissions from at least a portion of said target skin area; and one or both of (d1) a processor, including related circuitry, electrically connected to one or more of the antenna array and the temperature sensor array, to receive and/or process data and/or signals; and—(d2) a server and/or database electrically connected to one or both of the device and the processor for storing related information and data.

Further aspects of the invention are as disclosed and claimed herein.

### BRIEF DESCRIPTION OF THE DRAWINGS

FIG. 1a is a top plan view of an exemplary embodiment of the device of the present invention;

FIG. 1b is a bottom plan view of an exemplary embodiment of the device of the present invention;

FIG. 1c is cross-sectional view of an exemplary embodiment of the device of the present invention taken along line A-A of FIG. 1a;

FIG. 1d is an elevated perspective view of an exemplary embodiment of the device of the present invention;

FIG. 2a is a top plan view of another exemplary embodiment of the device of the present invention;

FIG. 2b is a bottom plan view of another exemplary embodiment of the device of the present invention;

FIG. 2c is cross-sectional view of another exemplary embodiment of the device of the present invention taken along line B-B of FIG. 2a;

FIG. 2d is an elevated perspective view of another exemplary embodiment of the device of the present invention;

FIG. 2e is an elevated perspective view of another exemplary embodiment of the device of the present invention;

FIG. 3a is an elevated perspective view of another exemplary embodiment of the device of the present invention;

FIG. 3b is a side plan view of an exemplary support of the device of the present invention as depicted in FIG. 3a; and

FIG. 3c is a bottom plan view of an exemplary support of the present invention as depicted in FIG. 3a; and

FIG. 3d is a perspective view of the exemplary embodiment of the device of the present invention as depicted in FIG. 3a and as deployed on a human skin surface.

### DETAILED DESCRIPTION

In one aspect, the method of the present invention may be a method for determining a mammalian vascular blood flow metric. The method of the present invention may include the steps of (a) heating a target skin area by irradiating said target skin area for a heating test period with radio frequency energy, said energy comprising a plurality of radio frequency beams; (b) measuring the temperature of said target skin area periodically during a measurement period to create a temperature data set; (c) determining at least one temperature change time constant based on the temperature data set; and (d) calculating a blood flow metric correlating to said temperature change time constant.

As used herein, the phrase “blood flow metric”, is intended to define or describe any numeric measure that may indicate, describe, represent or qualitatively or quantitatively correlate to blood flow in a mammalian body, for example in vascular blood vessels. Blood flow may include for example one or more of volume or mass flow, volume or mass blood flow rate per volume or mass of tissue; flow rate change or variation and rate of said flow rate change. Non-limiting examples of blood flow metric as described herein include temperature time change constants such as heating time constant, cooling time constant and total temperature change time constant.

A step of the present method includes heating a target skin area by irradiating the target skin area for a heating test period with radio frequency energy. The energy may include a plurality of radio frequency energy beams, generated for example by a plurality of antennas, such as for example multiple planar antennas directed at the same location or target skin area or portion thereof. In one or more embodiments, the radio frequency energy or the radio frequency energy beams may be spatially overlapping (including partially or totally overlapping) radio frequency energy beams. In general, the radio frequency energy and/or radio frequency beams are characterized by a frequency of from 100 KHz to 300 GHz. In one or more exemplary embodiments, the radio frequency energy or the radio frequency beams may be characterized by a frequency from 20 GHz to 40 GHZ. In one or more exemplary embodiments, the radio frequency energy or the radio frequency beams may be characterized by a frequency of from 20 GHz to 300 GHz. In one or more exemplary embodiments, the radio frequency energy or the radio frequency beams may be characterized by a frequency of from 6 GHz to 300 GHz.

In one or more exemplary embodiments, the radio frequency energy or said radio frequency beams may be a phase-shifted plurality of spatially overlapping radio frequency beams. As used herein, the term “phase shifted” is defined as or intended to describe a time offset applied to the generation of radio frequency energy or said radio frequency beams from their respective antennas or transmitter, with the time offset translating to a distance corresponding to the spatially overlapping radio frequency beams' wavelength or some portion thereof. In one or more exemplary embodiments, the radio frequency energy or said radio frequency beams may be phase-shifted an amount of up to ±½ of the wavelength of the spatially overlapping radio frequency beams. A preferred phase-shift amount may vary depending on a number of factors, including without limitation antenna arrangement and spacing, specified operating frequency and antenna distance from target skin area. In one or more exemplary embodiments, the radio frequency energy or said radio frequency beams may be an in-phase plurality of radio frequency beams. In one or more exemplary embodiments, the radio frequency energy or said radio frequency beams comprises a plurality of pulsed radio frequency beams. In one or more exemplary embodiments, the radio frequency energy or said radio frequency beams comprises a plurality of pulsed radio frequency beams with a duty cycle of from 0.10 to 0.90, with “duty cycle” defined as the percentage of time the antenna is transmitting over a total reference time period versus the total reference time period. In one or more exemplary embodiments, the radio frequency energy comprises a plurality of continuous wave (CW) radio frequency beams. In one or more exemplary embodiments, the radio frequency energy or said radio frequency beams comprise pulsed radio frequency beams or continuous wave (CW) radio frequency beams.

In the present method, radio frequency energy irradiates a target skin area, which in general causes an increase in the temperature of the skin in the target area. The amount and rate of the temperature increase may depend on a number of variables, including without limitation radio frequency energy or beam power, frequency, wavelength and angle of incidence; distance of irradiation source from the target skin area; irradiation time period; and the dimensions of target skin area. The target skin area may also be characterized by an associated absorption coefficient, which may be generally described as a fraction of the incident energy that is absorbed by the medium of the target skin area surface and which can vary with the frequency of the wave. Further, the penetration depth and the absorption coefficient are functions of the wave frequency and may be affected by various properties of the targeted skin, including but not limited to skin composition, hydration, fat, and salt content and distribution, presence and distribution of hair follicles, sweat glands and other adnexa. Penetration depth is the depth at which (i) the power density has decreased to 14% of the power density incident at the surface or (ii) the electric field magnitude has decreased to 37% of the electric field magnitude incident at the surface.

Generally, the penetration depth of human skin is on the order of a few millimeters or less at RF frequencies in the range of 10 GHz-100 GHZ, with penetration depth generally decreasing with increasing frequency. Though not in any way limiting the present invention, the angle of incidence of the wave may be equal to or less than 90 degrees with respect to the plane of the target or the angle of incidence may be equal to or greater than a zenith angle of zero degrees; the target area may be generally planar; and the incident power density (a function of the wave forward power, the dimensions of the target area and the distance from the wave source to the target) may be less than about 1 W/cm² normal to the target surface. In general, these variables may be managed to cause an average skin temperature increase rate of no more than 0.1 degrees Centigrade per second. It is preferred that target skin area temperature generally not exceed 43 degrees Centigrade during the irradiating step or during the method.

The step of (a) heating a target skin area by irradiating said target skin area with radio frequency energy extends for a heating test period. In general, a heating test period may be described as any time period during which the target skin area is being heated in step (a). In one or more exemplary embodiments, the heating test period may begin at commencement of heating, with the temperature of the target skin area at some selected or desired initial condition, such as for example at or near normothermic, and extend at least partially or completely through achievement of a “plateau temperature”, defined as a temperature which does not change appreciably over successive measurements, with “appreciably” describing a measurement change greater than the uncertainty of the temperature measurements.

In a period subsequent to the heating step, during which radio frequency energy irradiation is not present and heating does not occur, the temperature of the target skin area may decline toward normothermic temperature. Such a period may be referred to herein as a cooling step or stage insofar as the temperature of the target skin area is generally decreasing.

In one or more exemplary embodiments of the method of the present invention, a step including one or more of (i) cleaning the target skin area, such as for example with alcohol or other agent(s) and/or (ii) removing hair from the target skin area, such as by shaving or depilation, may precede heating step (a).

The method of this aspect of the present invention includes a step (b) of measuring the temperature of said target skin area periodically during a measurement period to create a temperature data set. The temperature measured in the measuring step may be referred to herein from time to time as a target skin area temperature. In one or more exemplary embodiments, the measuring step includes receiving the electromagnetic energy (in some embodiments, infrared energy) emission from the target skin area, such as for example with or using an infrared temperature sensor array that includes a plurality of infrared temperature sensors. Other known and suitable temperature measuring or sensing devices, including for example one or more of transducers, pyrometers, bolometers, fiber optic thermometers, infrared thermography cameras or other components generally operable to sense a temperature of the target skin area, may also be useful alone or in combination with each other or with infrared temperature sensors for performing the measuring step. In one or more exemplary embodiments, the measuring step includes receiving two or more different wavelengths or two or more overlapping or non-overlapping wavelength bands of infrared energy emission from the target skin area, for example by using a sensor array with infrared temperature sensors that detect two or more different infrared wavelengths or two or more overlapping or non-overlapping infrared wavelength bands.

The measuring of step (b) may occur periodically during a measurement period. In general, the measurement period may be any time period during which temperature measurement readings are taken under measuring step (b). In one or more exemplary embodiments, the measurement period may run concurrent with or be within the heating test period. In one or more exemplary embodiments, the measurement period begins prior to or at the beginning of, and extends into, the heating test period. In one or more exemplary embodiments, the measurement period may begin at the end of or after the heating period or may extend at least partially during the cooling period described above. In one or more exemplary embodiments, the measurement period begins during the heating test period and extends through and past the end of the heating test period. The term “periodically” is intended to include any set of multiple temperature measurements or readings taken over the measurement period, including those taken (i) at the beginning and/or end of the measurement period; (ii) at regular intervals during the measurement period; (iii) manually, on demand or more generally at irregular intervals; or any combination thereof, during the measurement period. In one or more exemplary embodiments, regular intervals may include without limitation from 1 reading per 5 seconds to 100 readings per second, preferably 10 readings per second to 100 readings per second.

The measuring of step (b) may create a temperature data set. As used herein, the phrase “temperature data set” is intended to describe the temperature readings taken during the measuring step (b) and any data or related information derived therefrom or useful for their understanding or interpretation, expressly including for example the time data corresponding to each temperature reading and/or the time intervals between readings. In one or more exemplary embodiments, the temperature data set expressly includes without limitation temperature readings taken from an infrared temperature sensor array or a plurality of infrared temperature sensors that comprise such an array.

The method of this aspect of the present invention includes a step (c) of determining at least one temperature change time constant based on the temperature data set. In general, the temperature change time constant may be described as the basis for the blood flow metrics calculated in step (d) discussed below and, though labeled as a “constant”, will typically range from 1 sec. to 100 sec., with variation depending on factors such as (without limitation) blood flow/unit area to the region, regional hyperemic response, capillary density and anatomy. In describing the present invention, the temperature change time constant may be generally determined from a temperature data set corresponding to a net temperature change in the target skin area. In general, the temperature change time constant t (tau) may be derived from the equation below:

$$T ( t ) = ( {T}_{2} - {T}_{1} ) \cdot ( 1 - {e}^{- t / τ} ) + {T}_{1}$$

Wherein T₁ and T₂ are target skin area temperatures at endpoints of the temperature data set and T(t) is a temperature reading of the target skin area at time t from the temperature data set. This presumes the measurement time period is sufficiently long such that the surface temperature has reached equilibrium or plateau temperature, i.e. the temperature is no longer changing appreciably with time.

In one or more exemplary embodiments, the temperature change time constant may be labeled as a heating time constant and may be determined based on a temperature data set corresponding to heating of or net temperature increase in the target skin area. In such embodiments, the heating time constant τₕ may be derived from the equation below:

$$T ( t ) = {T}_{initial} + ( {T}_{max} - {T}_{inital} ) \cdot ( 1 - {e}^{- t / {τ}_{h}} )$$

wherein Tₘₐₓ is the plateau temperature of the target skin area; Tᵢₙᵢₜᵢₐₗ is the initial temperature of the target skin area at the start of the heating period; and T(t) is the skin surface temperature reading at time t from the temperature data set.

In practice the time constant is obtained by fitting the temperature data to the equation

$$T ( t ) - {T}_{initial} = Θ ( 1 - {e}^{- t / {τ}_{h}} )$$

where both Θ and τₕ are determined using known data set modeling or curve-fitting techniques.

In one or more exemplary embodiments, the temperature change time constant may be labeled as a cooling time constant and may be determined based on a temperature data set corresponding to cooling of or net temperature decrease in the target skin area. In such embodiments, the cooling time constant τ_{c} may be derived from the equation below:

$$T ( t ) - {T}_{initial} = - Θ ( 1 - {e}^{- t / {τ}_{c}} )$$

wherein Tᵢₙᵢₜᵢₐₗ is the surface temperature of the target skin area at the start of the cooling phase, which in some embodiments may be the plateau temperature; and T(t) is the temperature reading at time t from the temperature data set. The parameters Θ and τ_{c} are determined using known data set modeling or curve-fitting techniques.

In one or more exemplary embodiments, the temperature change time constant may be labeled as a total temperature change time constant and may be determined based on a temperature data set corresponding to both heating or net temperature increase in the target skin are and cooling of or net temperature decrease in the target skin area. In such embodiments, the total time constant Ttot may be derived from a combination of the heating and cooling time constants:

$${τ}_{tot} = f ( {τ}_{h} ) + g ( {τ}_{c} )$$

Wherein ƒ(τₕ) and g(τ_{c}) are functions of the heating and cooling time constants, respectively. The time constants and related parameters described above may be derived from or using the corresponding equations using known and conventional data set modeling or curve-fitting methods. Non-limiting examples include least squares fit, nonlinear regression, Gauss-Newton method, Levenberg-Marquardt algorithm and the like. A preferred derivation technique includes least-squares fit. Suitable mathematical software packages for least-squares fit and other derivation methods are also known and commercially available and may be exemplified by Prism, Matlab, and Octave.

In a non-limiting example of a least-squares fit, the time constant t can be obtained by applying a least-squares fit of the temperature measurements, taken over some time interval, to the model. In this example the data will be fit to the temperature model T(t)−T₀=A·(1−e^{-t/τ}), where T₀ is the temperature at time t=0 and T(t) is the skin surface temperature at time t. The values of parameters A and τ are to be determined from the least-squares fit. In one non-limiting example, assume 100 temperature measurements, taken at constant intervals. The temperature measurements are designated Tᵢ, where i has values from 1 to 100, while the corresponding time value for each measurement is denoted by tᵢ. Values of τ and A can be determined by minimizing the value of the sum of the squares of the residuals S with respect to τ and A

$$S = {\sum }_{i = 1}^{N} {[ {T}_{i} - A ( 1 - {e}^{- {t}_{i} / τ} ) ]}^{2}$$

where the summation function

$${\sum }_{i = 1}^{N} f ( {t}_{i} )$$

is defined as the sum of all values of the function ƒ(tᵢ) from i=1 to i=N, and tᵢ is the time corresponding to the i^{th} temperature measurement.

In this example, N=100, corresponding to the number of temperature values (and corresponding time values.) In this example, the temperature data are fit to the model using unweighted residuals rᵢ, where

$${r}_{i} = {T}_{i} - A ( 1 - {e}^{- {t}_{i} / τ} )$$

In some cases a weighting function may be employed to reflect differing levels of uncertainty in the measured values.

The method of this aspect of the present invention includes a step (d) of calculating a blood flow metric, described herein using the convention of a letter “M”. In one or more embodiments, the blood flow metric may be dimensionless. As noted above, a blood flow metric may be described as any numeric measure that may indicate, describe, represent or qualitatively or quantitatively correlate to blood flow in a mammalian body. In one non-limiting example, the step (d) includes calculating a blood flow metric correlating to a temperature change time constant as described above.

In one or more exemplary embodiments, step (d) includes calculating a blood flow metric M according to the equation

$$M = C \cdot τ + D$$

where τ is determined as described above; C is a conversion coefficient selected for example to generate numeric values for M that are more suitable or convenient for clinical use and analysis; and D is an optional bias term useful for example to generate values more suitable or convenient for clinical use and analysis. In embodiments where τₕ is determined from the heating phase measurements, a blood flow metric Mₕ may be determined according to the equation.

$${M}_{h} = A \cdot {τ}_{h} + E$$

where τₕ is determined as described above; A is a conversion coefficient selected for example to generate numeric values for Mₕ that are more suitable or convenient for clinical use and analysis; and E is an optional bias term useful for example to generate values more suitable or convenient for clinical use and analysis. Similarly, in embodiments where τ_{c} is determined from cooling phase measurements, the blood flow metric Mc may be determined according to the equation

$${M}_{c} = B / {τ}_{c} + F$$

where τ_{c} is determined as described above; B is a conversion coefficient selected for example to generate numeric values for Mc that are more suitable for convenient for clinical use and analysis; and F is an optional bias term useful for example to generate values more suitable or convenient for clinical use and analysis.

In one or more exemplary embodiments, a blood flow total metric may be determined according to the equation

$${M}_{t} = A \cdot {τ}_{h} + B / {τ}_{c} + G$$

where τₕ and τ_{c} are determined as described above; A and B are conversion coefficients selected for example to generate numeric values for Mₜ that are more suitable or convenient for clinical use and analysis; and G is an optional bias term useful for example to generate values more suitable or convenient for clinical use and analysis.

As a general matter, units for conversion coefficients and bias terms in each of the above equations may be selected such that a resulting blood flow metric may be dimensionless and may therefore be different between specific calculations. Conversion coefficients and bias terms may individually and collectively be referred to herein as calculation factors.

In one non-limiting example, conversion coefficients A and C might be assigned a value of 1000 sec⁻¹ and B might be assigned a value of 1 sec in instances where time constant values, with units of seconds, are of relatively small orders of magnitude such as hundredths or thousandths and/or carry a large number of significant figures. Typical but non-limiting time constant values, in order of magnitude, may be in the tens of seconds or about 60 seconds (one minute). In one or more embodiments, the determining step (c) may be performed without reference to or use of power or power density data relating to the radio frequency energy or the radio frequency beams. In one or more embodiments, the calculating step (d) may be performed without reference to or use of power or power density data relating to the radio frequency energy or the radio frequency beams. In one or more embodiments, the determining step (c) and the calculating step (d) may be performed without reference to or use of power or power density data relating to the radio frequency energy or the radio frequency beams.

In one or more embodiments, conversion coefficients and bias terms may be values learned using for example artificial intelligence or machine learning techniques, such that conversion coefficients may be learned conversion coefficients and bias terms may be learned bias terms. Accordingly, the calculating step (d) of the method of the present invention may include establishing values for at least one of conversion coefficients and bias terms using artificial intelligence or machine learning. Non-limiting examples of useful artificial intelligence and machine learning techniques are known in the art and include logistic regression, linear discrimination analysis (LDA) and the usage of machine learning with simple linear classifiers such as the perceptron. In such techniques, training data associated with the classification scenario may be used to derive the conversion coefficient and/or bias term values and a logistic function employed to derive the probability value for the corresponding classification threshold. This simple logistic model may be improved by the usage of LDA to determine the linear boundary to maximize class separation (e.g., normal versus abnormal or impaired blood flow). In a simple form, the derivation of the classification metric and subsequent classification could be performed by the perceptron (McCulloch-Pitts neuron), where training is used to derive the coefficients and bias terms associated with a simple linear threshold function. Threshold optimization may be achieved through several methods including manual threshold sweeping, hyperparameter optimization frameworks or genetic algorithms. Elements to perform such classification approaches, and more generally elements useful to establish values for calculation factors, may be referred to as “artificial intelligence elements”. In some embodiments, artificial intelligence elements may be components of the system of the present invention described in detail below. In some embodiments, artificial intelligence elements may be integrated into or a component of the device of the present invention described in more detail below. Artificial intelligence elements may provide output in real time or may be utilized in offline analyses subsequent to data collection.

It will be appreciated that the methods of the present invention may be useful in supporting research, health assessment and diagnoses relating to a variety of mammalian species, including both humans and animals, and therefore may be used by medical professionals, veterinarians, researchers and the like across numerous disciplines. In one non-limiting example, the method and device of the present invention may be useful in assessing blood flow changes in response to stimuli such as drug administration, vascular occlusion, revascularization therapy or thermal stimuli.

It will be appreciated that the methods of the present invention generate information, data and related blood flow metrics that can be collected and qualitatively or quantitatively analyzed to assess overall health and fitness of a medical patient as well as more specific medical phenomena in conjunction with risk assessment, patient monitoring, therapeutic vascular intervention and medical condition diagnoses. Accordingly, in one or more exemplary embodiments, a mammalian vascular blood flow rate may be determined using or with reference to the mammalian vascular blood flow metric obtained by the method, device or system of present invention. Further, the method may be employed or performed multiple times. In one or more exemplary embodiments, the method may be repeated two or more or three or more times. In one or more exemplary embodiments, the method may be repeated two or more or three or more times in sequence utilizing the same target test area. In one or more exemplary embodiments, the method may be repeated two or more or three or more times concurrently utilizing multiple target test areas. In one or more exemplary embodiments, the method may be repeated two or more or three or more times sequentially using multiple target test areas.

One of ordinary skill will appreciate that the method steps of the present invention may be partially or wholly performed, facilitated or assisted using devices or systems capable of performing the method steps described above. In one example, the method of the present invention may be performed using the device of the present invention or a system of the present invention as described herein. Accordingly, descriptions and disclosures relating to elements or features of an aspect or embodiment of the present invention are hereby expressly relied on to describe and support those elements or features in other aspects or embodiments. More particularly, descriptions and disclosures relating to elements or features of the method for determining a mammalian vascular blood flow metric are hereby expressly relied on to describe and support those elements or features in the device for determining a mammalian vascular blood flow metric and the system for determining a mammalian vascular blood flow metric described herein, and vice versa.

In another aspect, the present invention relates to a device for determining a mammalian vascular blood flow metric at a target skin area 100. As shown for example in the Figures, the device 10 of the present invention may include: a. at least one support 20; b. an antenna array 40 including a plurality of radio frequency beam-emitting antennas 45 mounted on support 20, such as for example on antenna mounting surface 25, and aligned to direct radio frequency energy at and heat said target skin area; and c. a temperature sensor array 50 comprising a plurality of temperature sensors 55 mounted on support 20, such as for example on temperature sensor mounting surface 30, and aligned to receive electromagnetic emissions (for example infrared emissions) from the target skin area 100. In one or more embodiments, sensors 55 or sensor array 50 measure the temperature of the target skin area and therefore are aligned to measure the temperature of the skin target area 100. The antenna array 40 and the temperature sensor array 50 (a) may be mounted in fixed relation to or a fixed distance from said target area and preferably (b) do not contact the target skin area 100. In one or more exemplary embodiments, the support 20 may further include at least one skin contacting surface 22 that contacts and is supported by skin surface 90 when the device is in use.

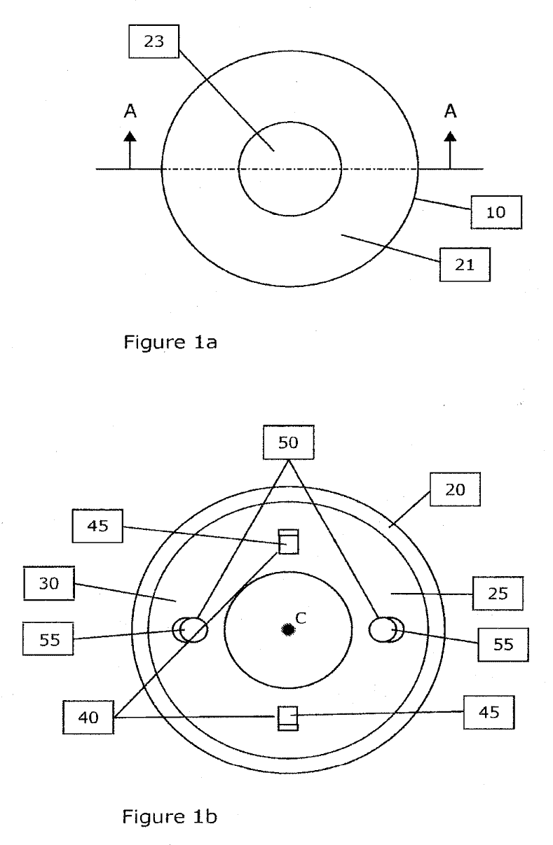

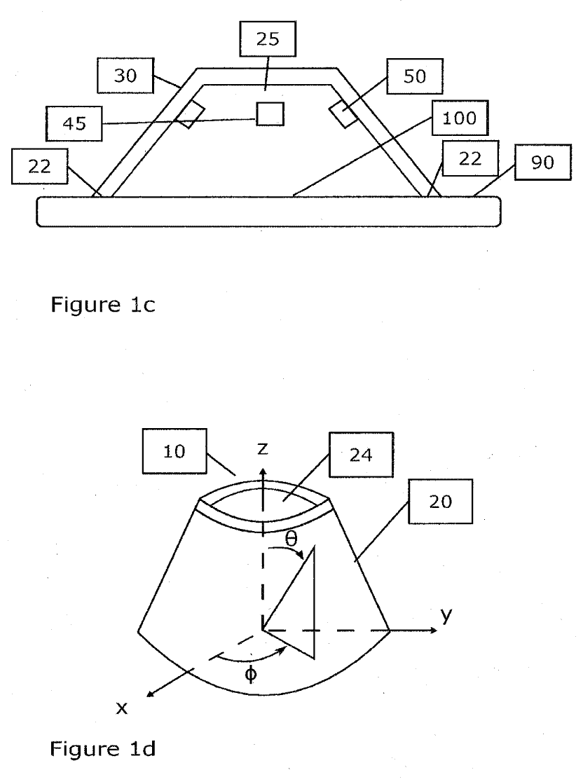

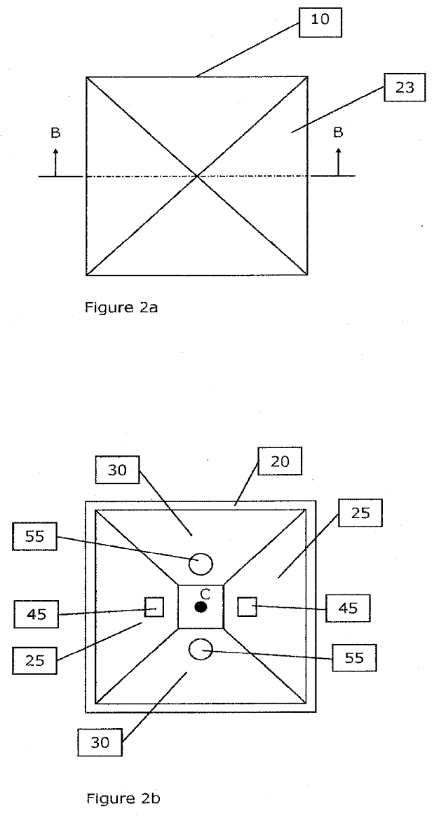

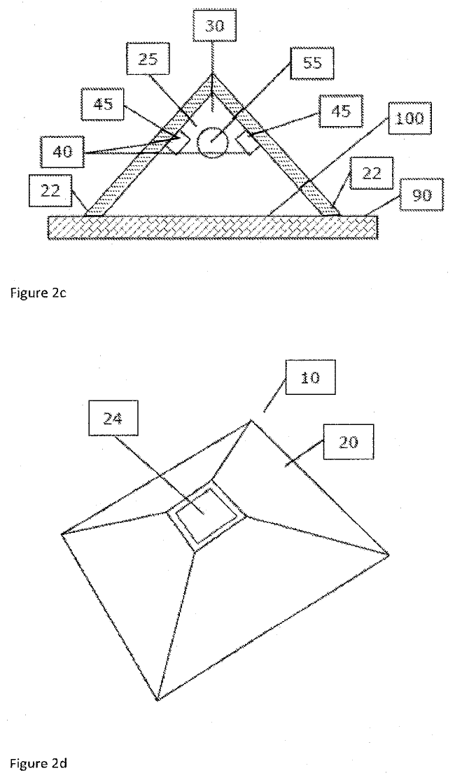

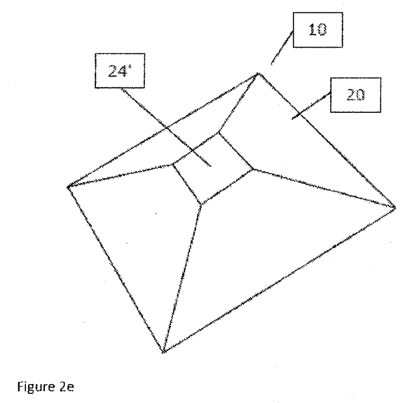

The support 20 may generally be configured in any way to achieve, and may be formed from materials suitable for, establishing and maintaining the antenna array and temperature sensor array in a fixed positional relationship to the target skin area. In one or more embodiments, suitable materials should not disrupt, absorb, reflect or otherwise impact electromagnetic energy waves and/or related electromagnetic fields related to device function. Suitable shapes and sizes for the support 20 may include without limitation frustoconical, conical, pyramidal, hemispherical, wedge-shaped, box-shaped and similar constructions. In a non-limiting example depicted in FIGS. 1a-1c, support 20 is frustoconical in shape and includes a common internal surface 21 that serves or functions as antenna mounting surface 25 and temperature sensor mounting surface 30; an antenna array 40 including two opposing radio frequency beam-emitting antenna 45; a temperature sensor array 50 including two opposing temperature sensors 55. In another non-limiting embodiment depicted in FIGS. 2a-2c, support 20 is pyramidal in shape and includes antenna mounting surfaces 25 and temperature sensor mounting surfaces 30; an antenna array 40 including two opposing radio frequency beam-emitting antenna 45; a temperature sensor array 50 including two opposing temperature sensors 55. “Opposing” is intended to describe two antennas or sensors whose line of intersection generally passes through a support center point C as depicted in the Figures.

One of ordinary skill will appreciate that the shape and size of support 20 may vary widely and numerous configurations may be contemplated that achieve the desired result of establishing and maintaining the antenna array 40 and temperature sensor array 50 in a fixed positional relationship to the target skin area 100. By way of example, at least a portion of the top surface 23 of the frustoconical support 20 as depicted in FIGS. 1a-1c may alternatively include an opening or aperture 24 as depicted in FIG. 1d. Similarly, at least a portion of the top pointed end 23 of the pyramidal support 20 as depicted in FIGS. 2a-2c may alternatively include an opening or aperture 24 or may include a flat top surface 24′ as depicted in FIGS. 2d and 2e.

Antenna array 40 and temperature sensor array 50 are mounted on support 20, for example at antenna mounting surface 25 and temperature sensor mounting surface 30 respectively and are supported thereby. Techniques and systems for mounting of antennas and sensors to a surface may be known to one of ordinary skill and include mechanical connectors, hook/loop fasteners, adhesives, cements, epoxies and the like. In some embodiments, antennas 45 and sensors 55 may be mounted or housed in recesses or apertures formed in surfaces 25 and 30 to provide a flush-mount fit. Antenna array 40 is aligned to direct radio frequency energy, preferably in the form radio frequency beams that overlap partially or totally from a spatial basis or perspective, at a skin surface target area 100, with the radio frequency energy causing a heating or temperature increase of the skin at target area 100. Temperature sensor array 50 may generally be configured in any way to achieve, and aligned in a manner suitable for measuring, a desired temperature of the skin surface target area 100, for example by receiving electromagnetic energy (in some embodiments, infrared energy) emissions from the target area such as emissions from the skin within the target area 100. Antenna array 40 and temperature sensor array 50 (a) may be mounted so as to be in fixed positional or distance relationship to the target skin area 100 and preferably (b) do not contact the target skin area 100.

Antenna array 40 may be mounted on the antenna mounting surface 30 and may include a plurality of radio frequency beam-emitting antenna 45 mounted on the antenna mounting surface 30. The radio frequency energy or the radio frequency energy emitted from the antennas may be spatially overlapping and together form the radio frequency energy emitted by the antenna array 40. In one or more exemplary embodiments, the plurality of radio frequency energy beam-emitting antennas may emit a plurality of spatially overlapping superimposed beams. In one or more exemplary embodiments, the radio frequency energy beam-emitting antenna include or consist of multiple planar antennas directed at the target skin area 100. In one or more exemplary embodiments, the radio frequency energy beam-emitting antennas may be driven in-phase with each other or out of phase with respect to each other or phase-shifted. In one or more exemplary embodiments, the beam-emitting antennas emit beams phase-shifted by an amount of up to ±½ of the wavelength of the spatially overlapping radio frequency beams. A preferred phase-shift amount may vary depending on a number of factors, including without limitation antenna arrangement, orientation and spacing, specified radio frequency energy/beam frequency, and distance from the target skin area. In one or more exemplary embodiments, the plurality of electromagnetic energy beam-emitting antennas may emit a plurality of spatially overlapping beams of a frequency in the range of 10 GHz to 40 GHz or from 10 GHz to 300 GHz or from 6 GHz to 300 GHz. In one or more exemplary embodiments, the plurality of electromagnetic energy beam-emitting antennas emit radio frequency energy in the form of a plurality of spatially overlapping radio frequency (RF) beams.

Antennas 45 of antenna array 40 may be mounted on antenna mounting surface 30 at a common fixed distance from the target skin area 100. Antennas 45 of antenna array 40 may be mounted on antenna mounting surface 30 and at a common, fixed zenith angle θ with respect to the target skin area 100. Antennas 45 of antenna array 40 may be mounted on antenna mounting surface 30 symmetrically with respect to the azimuth or azimuthal angle φ. As known in the art, azimuth is the angle formed between a reference direction and a line from the observer to a point of interest projected on the same plane as the reference direction orthogonal to the zenith. Zenith is the imaginary point on the celestial sphere directly “above” (in a vertical direction) a particular location. A general depiction for azimuthal angle ¢ and zenith angle θ is provided for reference at FIG. 1d. Each antenna 45 of antenna array 40 may be individually oriented in TE (transverse electric) polarization or TM (transverse magnetic) polarization and the antennas in antenna array may accordingly be oriented in TE (transverse electric) polarization, TM (transverse magnetic) polarization or a combination thereof.

Temperature sensor array 50 includes in general one or more devices or sensors 55 suitable for non-contact measurement of the temperature of a surface. Suitable examples include devices such as are commercially available from Melexis, Inc. under the trade designation MLX90614. In one or more exemplary embodiments, the temperature sensor array 50 may include a plurality of temperature sensors 55. In one or more exemplary embodiments, the temperature sensor array 50 may include one or more of or a plurality of temperature sensors 55 receiving infrared emissions. In one or more exemplary embodiments, the temperature sensor array 50 may include a plurality of temperature sensors 55 receiving electromagnetic emissions or infrared emissions of at least two different wavelengths or wavelength ranges or bands. In one or more exemplary embodiments, the temperature sensor array 50 may include a plurality of infrared temperature sensors 55. In one or more exemplary embodiments, the temperature sensor array 50 may include a plurality of temperature sensors 55 receiving electromagnetic emissions of at least two different wavelengths or at least two different wavelengths (bands) in the infrared region, with “infrared” region as used herein intended to extend generally from about 0.7 μm to about 1000 μm inclusive.

Insofar as the device requires electrical power to operate, the device may further include a power supply, for example an onboard battery or battery pack to provide DC (direct current) to the device at the required voltage(s). Suitable power supplies may be external (as exemplified by AC power supplied by a building plug and transferred to the device via wired connection with rectification and/or voltage regulation as necessary) or preferably are internal to or integral with the device (as exemplified by a battery pack mounted on the device, again with voltage regulation as necessary). Batteries may be either rechargeable or non-rechargeable. Power supplies are to include appropriate protections against overheating and short-circuits. External power supplies are to have protection against ground faults.

The device may also include or incorporate one or more operational features, including for example features related to device, user or operator safety. In one example, the device may include a temperature limiter. A temperature limiter may include hardware, cooling devices or systems such as heat sink(s), cooling fan(s), heat pipe(s), extended surfaces, fins or the like or related components to prevent or reduce risk of device overheating which may cause serious damage to the device. Temperature limiters may include hardware to sense the temperature of the device or device components, in particular the device electronics such as circuit boards or RF amplifier which may generate substantial heat when in use, and dissipate heat therefrom.

In one example, the device may include a skin protection auto shut-off mechanism. Skin protection auto shut-off may typically include software, hardware or similar components to shut off the device and/or disable its power supply should one or more of the temperature sensors of the temperature sensor array detect a target skin area temperature exceeding a predefined temperature threshold. As discussed above, the target skin area temperature should generally not exceed 43° C. Accordingly, in some embodiments, the skin protection auto shut-off may include a predefined temperature threshold of 43° C. or less. In some embodiments, the skin protection auto shut-off may include a predefined temperature threshold of 40° C. or less to provide an additional margin of safety. The skin protection auto shut-off is an important personal safety feature as it protects the patient/subject from possible temperature-induced skin injury such as burns or blistering in the event of a malfunction

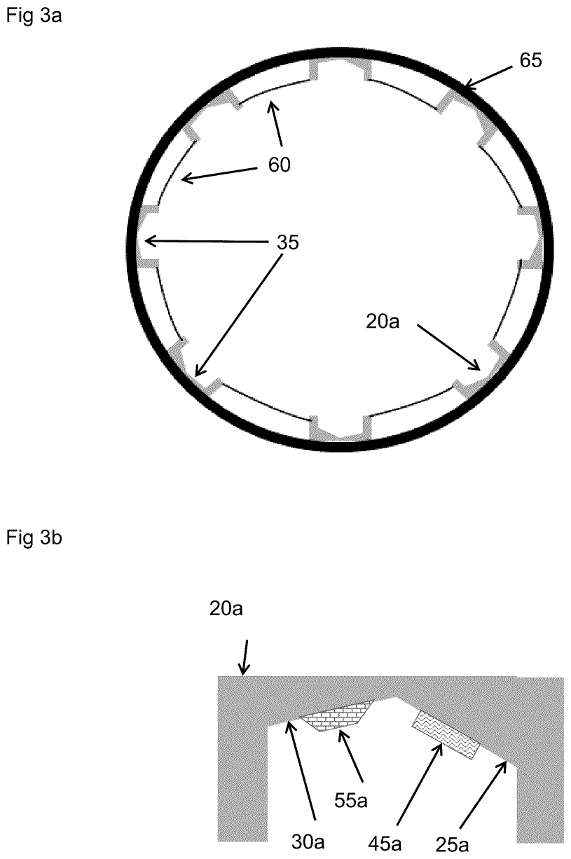

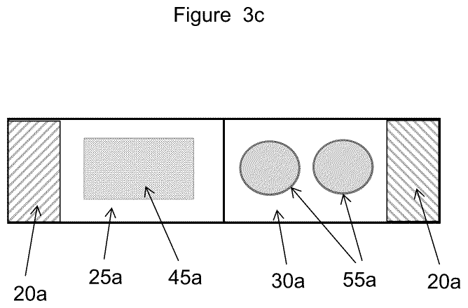

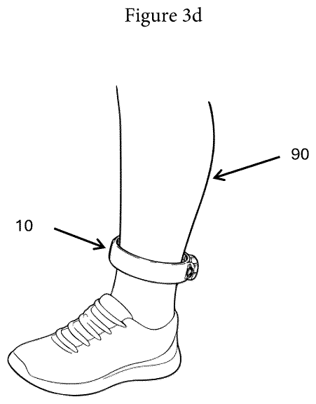

In one or more exemplary embodiments, the device may include two or more supports. An example of such an embodiment is depicted in FIGS. 3a, 3b and 3c. With reference to these Figures, the device 10 of the present invention may include two or more spaced individual supports 20a, which collectively may be referred to as a support assembly 35. The support assembly 35 may include two or more or three or more or four or more or five or more or six or more spaced individual supports 20a with each individual support 20a traversing at least a portion of the skin target area 100 . . . . Each support 20a may include an antenna mounting surface 25a on which one or more antennas 45a may be mounted and a temperature sensor mounting surface 30a on which one or more temperature sensors 55a may be mounted. For purposes of exemplary illustration only, the device depicted in FIG. 3b includes a support assembly 35 with eight spaced individual supports 20a. It is, however, to be understood that the device of the present invention is not limited to the number depicted in the Figures or to any particular number.

In one or more exemplary embodiments that include a support assembly 35 with two or more spaced individual supports 20a, the device 10 of the present invention may further include stabilizers 60 extending between and/or connecting adjacent spaced individual supports 20a. Stabilizers 60 may serve to maintain device and device component position and alignment, for example with respect to supports 20a, the skin surface 90 and target skin area 100 or portion thereof. Stabilizers 60 may include or be formed from any suitable material, as exemplified by elastic or inelastic webs, strips, or similar forms of films, woven or non-woven fabrics or the like constructed from polymers, plastics or organic material.

In one or more exemplary embodiments, the device of the present invention may include a carrier on or in which support 20, two or more supports 20a or support assembly 35 may be mounted. An exemplary embodiment that includes a carrier 65 is depicted at FIG. 3c. In this exemplary embodiment, carrier may be a band or similar construction suitable for circumferential deployment or installation on for example a human appendage such as an arm or leg or a portion thereof such as a calf, ankle, thigh, forearm or wrist. Carrier may be formed for example as a tube from an elastic fabric and be deployed by applying force to stretch or expand the carrier over the appendage and releasing the applied stretching force once the device is located in the desired position on skin surface 90. Carrier may be formed from a rectangular piece of fabric with an optional conventional fastener system such as a hook-and-loop, pin- and hole, snap, button or zipper fastener pairings at its respective ends and be deployed by wrapping the carrier around the appendage at the desired position on skin surface 90 and connecting the respective fastener pairing portions. Carrier may be formed from a fabric or polymer film with an adhesive outer boundary portion and may be deployed by adhesive attachment to the skin surface 90. Other configurations for carrier 65 may be within the scope of the ordinary skilled artisan.

The above-described device of the present invention may be a component of a system for determining a mammalian vascular blood flow metric that may include additional components to perform, facilitate and/or assist with various related or supporting functions such as device and device component control; data receipt, collection, transfer, processing and manipulation; calculation such as determination of metrics; information and data display; and/or similar functions. Accordingly, in another aspect, the present invention relates to a system for determining a mammalian vascular blood flow metric. The system may include (i) a device for determining a mammalian vascular blood flow metric at a target skin area, the device including a. at least one support including an antenna mounting surface and a temperature sensor mounting surface; b. an antenna array including a plurality of radio frequency beam-emitting antennas mounted on the antenna mounting surface and aligned to direct radio frequency energy at and heat at least a portion of said target skin area; and c. a temperature sensor array comprising a plurality of temperature sensors mounted on the temperature sensor mounting surface and aligned to receive electromagnetic emissions from at least a portion of the target skin area 100; and one or both of (d1) a processor, including related circuitry, electrically connected to one or more of the antenna array and the sensor array, to receive and/or process data and/or signals; and (d2i) a server and/or database electrically connected to one or both of the device and the processor for (by way of example) storing related information and data and performing calculations to support blood flow metric determination. In one or more embodiments, the temperature sensors or the temperature sensor array are aligned to measure the temperature of the target skin area.

The processor may be for example a computer, tablet, cellular or mobile phone, smart phone, onboard logic circuit or any other device. The processor may include I/O interfaces and storage devices or memory and may communicate and receive commands from external or integrated input devices.

The processor may include one or more software programs, the function and operation of which may be encompassed in suitable hardware components and/or modules. A software program may for example include (a) an acquisition module operable to acquire and receive data from one or more of the processor, the temperature sensor array and the antenna array; (b) an analysis module electrically connected to the acquisition module and operable to receive the data from the acquisition module and analyze the data; and (c) an output module electrically connected to the acquisition module and/or the analysis module and operable to receive data from the acquisition module, receive data from the analysis module, format the data, and output the data to a human readable form using for example a display, smart phone screen, printer, or similar output device. The analysis module may for example perform one or more mathematical functions, manipulate data, correlate data, format data, and/or perform other analytical functions.

The system may include a server and/or database for storing related information and data to perform calculations relating to blood flow metric determination. The server may include an operating system for running various software programs and/or a communications applications. The software programs may be manipulated by computer terminals (not shown) and/or medical equipment (not shown) to acquire, enter, review, and/or save information. The processor may interface with and/or connect to the server to upload and/or download information or print data and related results.

In one or more exemplary embodiments, one or more of the various elements or components of the system may be part of, integrated into or a component of the device of the present invention. In one or more exemplary embodiments, the entire system may be integrated with the device such that the device power supply, RF transmitter and amplifier, controller, sensor(s), antenna (e), processor and display are contained within a single enclosure. Said enclosure may incorporate materials such as copper, aluminum or other materials so as to avoid or reduce electromagnetic interference with surrounding devices and systems. The enclosure or system may also separately include or incorporate operational features, including without limitation temperature limiters and skin temperature auto shut-off mechanisms such as described above in the context of the device, for example to prevent or reduce risk of (i) overheating of the device and/or (ii) temperature-induced skin impact or injury such as pain, burns or blistering. In one or more exemplary embodiments, the device may include embedded software or firmware, for example to provide control and/or processing functionality.

In one or more exemplary embodiments, one or more of the various elements or components of the system may be separate from but communicate with the device of the present invention or the temperature sensor array or the antenna array. Communication between the various components or elements of the system may be established with wired connectivity or wireless protocols such as Bluetooth®. Accordingly, “electrically connected” elements may be connected by any known data or communication means, including but not limited to wired connections and wireless connections.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

That which is claimed is:

1. A method for determining a mammalian vascular blood flow metric, said method comprising the steps of (a) heating a target skin area by irradiating said target skin area for a heating test period with radio frequency energy, said energy comprising a plurality of radio frequency beams; (b) measuring the temperature of said target skin area periodically during a measurement period to create a temperature data set; (c) determining at least one temperature change time constant based on the temperature data set; and (d) calculating a blood flow metric correlating to said temperature change time constant.

2. The method of claim 1 wherein said radio frequency energy or said radio frequency beams comprise spatially overlapping radio frequency beams.

3. The method of claim 1 wherein said radio frequency energy or said radio frequency beams may be characterized by a frequency from 6 GHz to 300 GHz.

4. The method of claim 1 wherein said radio frequency energy or said radio frequency beams are phase-shifted an amount of up to +½ of the wavelength of said spatially overlapping radio frequency beams.

5. The method of claim 1 wherein said radio frequency energy or said radio frequency beams comprise an in-phase plurality of overlapping radio frequency beams.

6. The method of claim 1 wherein said radio frequency energy or said radio frequency beams comprise a plurality of pulsed radio frequency beams.

7. The method of claim 1 wherein said radio frequency energy or said radio frequency beams comprise a plurality of continuous wave (CW) radio frequency beams.

8. The method of claim 1 wherein said measurement period runs concurrent with or is within said heating test period.

9. The method of claim 1 wherein said measurement period begins prior to or at the beginning of, and extends into, said heating test period.

10. The method of claim 1 wherein the measurement period begins at the end of or after said heating test period.

11. The method of claim 1 wherein the measurement period begins during said heating test period and extends through and past the end of said heating test period.

12. A device for determining a mammalian vascular blood flow metric at a target skin area, said device comprising
a. at least one support;
b. an antenna array including a plurality of radio frequency energy beam-emitting antennas mounted on said support and aligned to direct radio frequency energy at and heat at least a portion of said target skin area; and
c. a temperature sensor array comprising a plurality of temperature sensors mounted on said support and aligned to receive electromagnetic emissions from at least a portion of said target skin area;
wherein said antenna array and said temperature sensor array are (a) mounted in fixed relation to said target skin area and (b) do not contact said target skin area.

13. The device of claim 12 wherein said radio frequency energy beam-emitting antennas comprise multiple planar antennas directed at said target skin area, with said antennas mounted at a common, fixed distance from the target skin surface.

14. The device of claim 12 wherein said the temperature sensor array comprises a plurality of temperature sensors receiving electromagnetic emissions of at least two different wavelengths or wavelength ranges in the infrared region.

15. The device of claim 12 further comprising one or both of a temperature limiter and skin protection auto shut-off mechanism.

16. A system for determining a mammalian vascular blood flow metric, said system comprising the device of claim 12.

17. The device of claim 12 wherein said support comprises a support assembly comprising two or more spaced individual supports, each traversing a portion of said target skin area.

18. The device of claim 17 further comprising stabilizers extending between adjacent spaced individual supports.

19. The device of claim 17 further comprising a carrier on which said support assembly is mounted.

20. The device of claim 19 wherein said carrier is a band for circumferential installation on a human or animal appendage.
